(19) United States
(12) Patent Application Publication
(10) Pub. No.: US 2021/0307688 A1
(43) Pub. Date: Oct. 7, 2021

(54) Skin Proximity Sensor for Tamper Detection

(71) Applicant: Milo Sensors, Inc., Santa Barbara, CA (US)

(72) Inventors: Bob M. Lansdorp, Santa Barbara, CA (US); Peter Lamberg, Santa Barbara, CA (US)

(21) Appl. No.: 17/301,395

(22) Filed: Apr. 1, 2021

Related U.S. Application Data
(60) Provisional application No. 63/003,836, filed on Apr. 1, 2020.

(51) Int. Cl. A61B 5/00

(52) U.S. Cl. CPC A61B 5/681; A61B 5/6832; A61B 5/0022; A61B 2562/0257; A61B 5/6831; A61B 5/4845; A61B 5/7246

(57) ABSTRACT

A means of detecting skin proximity of a wearable comprising: at least two skin proximity sensors, a controller and a transmitter. The proximity sensors can be arranged around a central biosensor to detect tamper attempts. The sensors can be strapped to a person's skin by means of a strap and further sealed on the skin by means of a bioadhesive. Signals from the proximity sensor can be processed by the controller and a ratio baseline obtained. Any appreciable deviations from unity will indicate a tampering event, such as device removal, sensor blockage or sabotage. Results of proximity sensing can be reported to the wearer by means of a monthly calendar, or a daily calendar view in a wearable device.

This application claims priority from provisional No. 63/003, 836, filed Apr. 1, 2020, the entire contents of which are herewith incorporated by reference.

This invention was made with government support under AA 026125, awarded by National Institute of Health (NIH). The government has certain rights in the invention.

### BACKGROUND

In many wearable sensor applications, it is important to detect whether or not a sensor is being worn on the intended person. Furthermore, it is desired to detect whether or not a system is tampered with, e.g. sensor blockage or removal. Transdermal alcohol sensors such as the SCRAM ankle bracelet have used an infra-red sensor (U.S. Pat. No. 7,462,149B2) combined with a locking ankle bracelet, to ensure that their transdermal alcohol sensor is being worn continuously, and not tampered with. However, the technology is bulky, indiscreet, and the sensor may not be capable of detecting tampers that circumvent the infra-red sensor.

For example, an individual might be able to defeat the sensing by placing a film of ethanol impermeable plastic which matches the reflectivity of the skin in between the skin and the sensor. Thus, a sufficiently motivated individual could consume alcohol while the sensor was reading zero alcohol.

Further, continuous monitoring on the same site of the body is known to cause skin irritation and chafing. Additionally, certain activities are not compatible with a bulky ankle bracelet that is not water-proof, such as swimming in the ocean, intense physical exercise, or meetings where discretion is of the utmost importance.

An existing approach to skin proximity detection US20130030320A1) used only a single skin-worn capacitive sensor to detect proximity changes, and compared the values to an internal reference. The use of a single skin-worn sensor capacitor will not be a reliable indication of tamper, since the natural fluctuations in capacitance during normal wear may exceed the typical fluctuations induced by tampering. Thus, what is desired is a more specific indicator of tampering.

In patent US20130030320A1, another embodiment is described with two capacitive sensors. However, in the description the invention sends the capacitive signals to a comparator, which greatly limits the ability to perform digital data analysis, since the data-rich raw data signals have been binarized to a simple on/off signal.

In another previous reference, US20160154952A1, capacitance is used to measure skin proximity. However, the inventors acknowledge that raw data is noisy, and propose to use a low-pass filter to alleviate the problem. Furthermore, the prior art leaves much to be desired in the signal to noise ratio of skin proximity detection and tamper detection.

### SUMMARY

Therefore, the inventor recognized the need for an improved system for tamper detection of an alcohol sensor. The inventor recognizes that an improved system for tamper detection is desirable.

The inventors also recognize the need for a system that enables a wearable to continuously monitor data, but which is removed for short periods of time without necessarily causing a tamper alert.

The present invention is related to the detection of the presence of a human being for continuous monitoring applications. The present application describes a human skin sensor, formed of at least two skin proximity sensors, whose values are compared to detect the presence of human skin and attempts to tamper.

### BRIEF DESCRIPTION OF THE DRAWINGS

FIG. 1 shows a skin proximity sensor having two electrodes on a wearable device;

FIG. 2 shows a two skin proximity sensors encircling an alcohol sensor on a wearable device;

FIG. 3 shows a schematic side-view of proximity sensors against the skin;

FIG. 4 shows a Skin-facing side of wearable skin proximity sensor;

FIG. 5 shows a Wrist-facing side of a skin proximity sensor;

FIG. 6 shows a cartridge with two capacitive sensor electrodes and an alcohol sensing electrode;

FIG. 7 show raw sensor data from two capacitive sensors;

FIG. 8 shows a ratio of two capacitive sensors during tampering events

FIG. 9 shows a flowchart of two proximity sensors

FIG. 10—flowchart describing how proximity sensor values can be used to detect a tamper event;

FIG. 11 shows a flowchart for a system for interrupted monitoring; and

FIG. 12 shows a Calendar view of tamper events.

### DETAILED DESCRIPTION

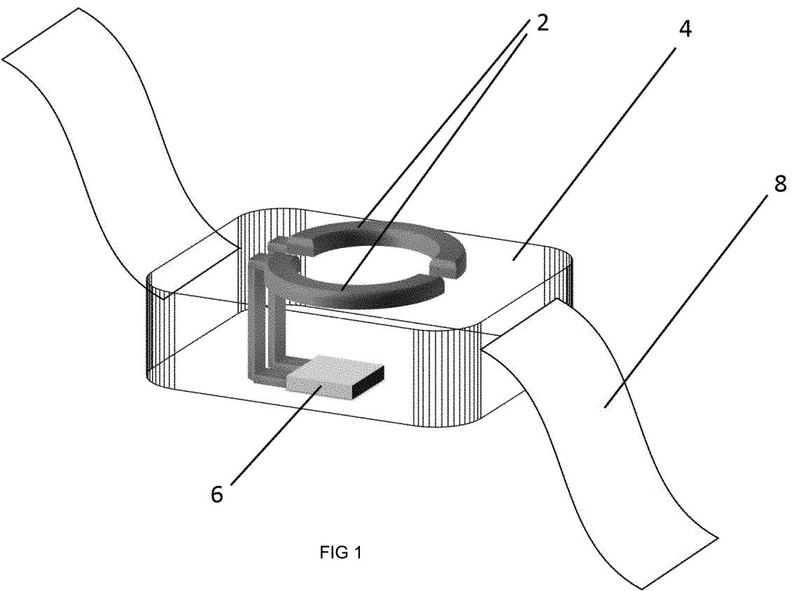

FIG. 1 illustrates an embodiment of a skin proximity sensor, intended to be wrist worn. In the FIG. 1 embodiment, first and second semi-circular skin proximity electrodes 2 are placed on a wrist wearable device housing 4. The electrodes 2 are connected to a microcontroller 6, which is placed inside the housing 4 and reads the capacitance of the electrodes 2.

The semi-circular skin proximity electrodes 2 are skin-facing and will make contact with human skin when the device is worn.

In one embodiment, the electrodes 2 are covered with a dielectric to reduce the noise of the signals. In another embodiment, the electrodes 2 are covered with a bioadhesive to improve the reliability of skin-contact.

The wearable device housing 4 has a strap 8, which can contain various means of fastening around a wrist or ankle, such as for example a tang-buckle clasp.

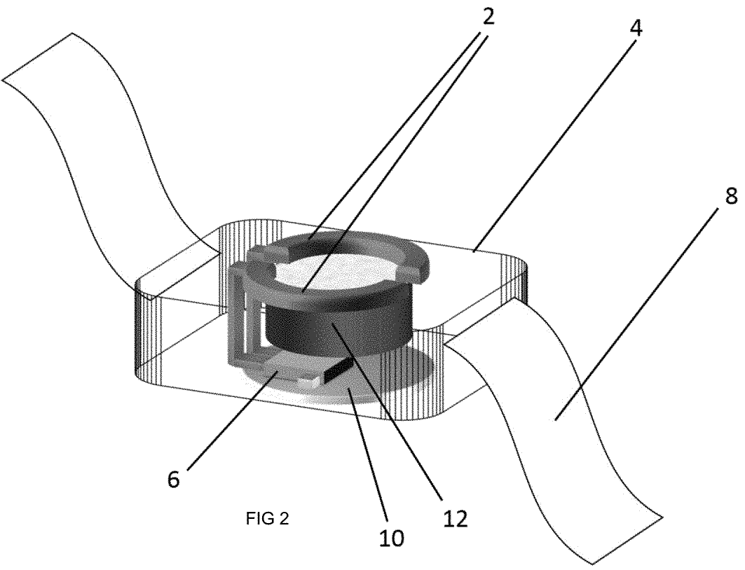

FIG. 2 shows an alternative embodiment, in which an additional ground plane 10 is added. This ground plane 10 could be connected directly to electrical ground of the microcontroller 6, or a driven shield of the microcontroller 6, with the function of shielding the sensor electrodes 2 from environmental electrical noise. The ground plane 10 can be co-planar with the other electrodes 2, or on a different layer, or be a hashed-ground patterned piece.

The ground plane 10 serves to reduce the electrical noise of the sensor and improve the quality of the resulting data. Further, a transdermal alcohol sensor 12 is added central to the two proximity electrodes 2. In this arrangement, the skin-proximity sensors 2 can detect any efforts to intentionally obstruct the transdermal alcohol sensor 12.

This transdermal alcohol sensor 12 could alternately be another sensor, such as for example an optical readout of blood oxygen, a measurement of sweat rate, or an optical measurement of heart-rate.

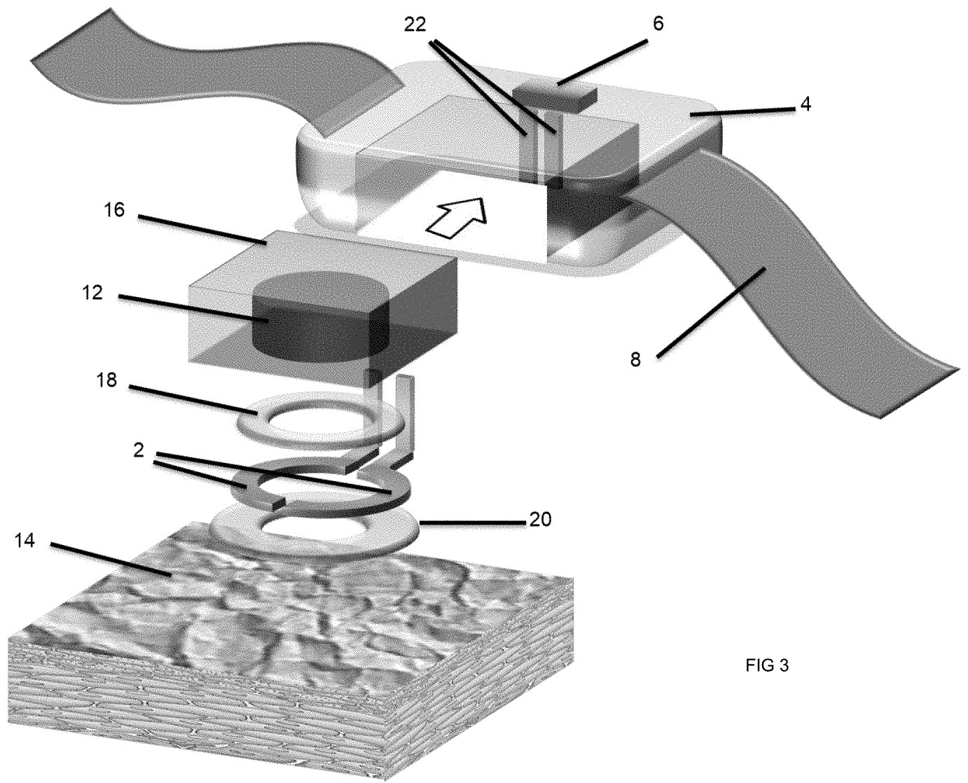

FIG. 3 shows a preferred embodiment of the invention. The wrist of a person is represented schematically as 14. The skin-proximity sensing electrodes 2 are attached to a cartridge 16 containing alcohol sensor 12, by means of an adhesive tape 18. The skin-facing side of the electrodes 2 are removably coupled to the skin of the person 14 by means of a bioadhesive layer 20. The bioadhesive could be acrylic, rubber, or a combination, but is preferable silicone bioadhesive on the skin-facing side and an acrylic on the other. The skin proximity sensors are arranged around a transdermal alcohol sensor 12. The cartridge 16 can be inserted into a housing 4 by a sliding mechanism or other means familiar to those skilled in the art. Skin-proximity sensing electrodes 2 are connected electrically to microcontroller 6 in housing 4 by means of contact pads 22, which can removably couple to the sensing electrodes.

The contact pads 22 and electrodes 2 could alternatively be connected by a press-fit, pogo-pins, or other means familiar to those skilled in the art

In the arrangement described in FIG. 3, attempts to tamper with the transdermal alcohol sensor 12, for example by inserting alcohol-impermeable plastic between the alcohol sensor 12 and the skin 14, will be detected by the proximity sensors 2. Transdermal alcohol vapors that are emitted from the skin make their way from the skin 14, between the proximity sensors, and into the sensor 12. In this embodiment, there are two advantages of sealing contact between skin and the device with bioadhesive 20. First, no alcohol vapors that are emitted from the skin will leak through gaps between skin and device, since the proximity sensor can detect tampering such as wristband removal, thus reducing false negatives in the reporting of alcohol consumption.

Secondly, any environmental alcohol, for example disinfectants or perfume containing alcohol that is placed near the sensor, does not make its way into the sensor, thus eliminating false positives.

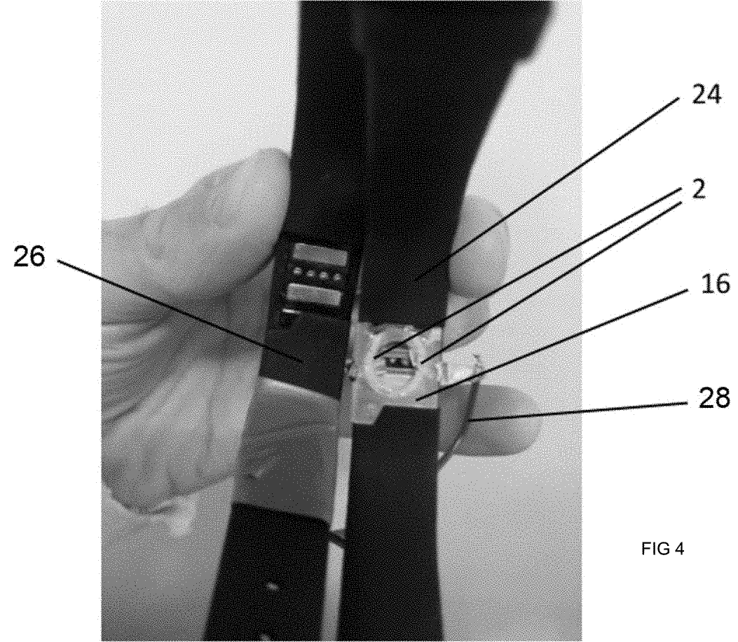

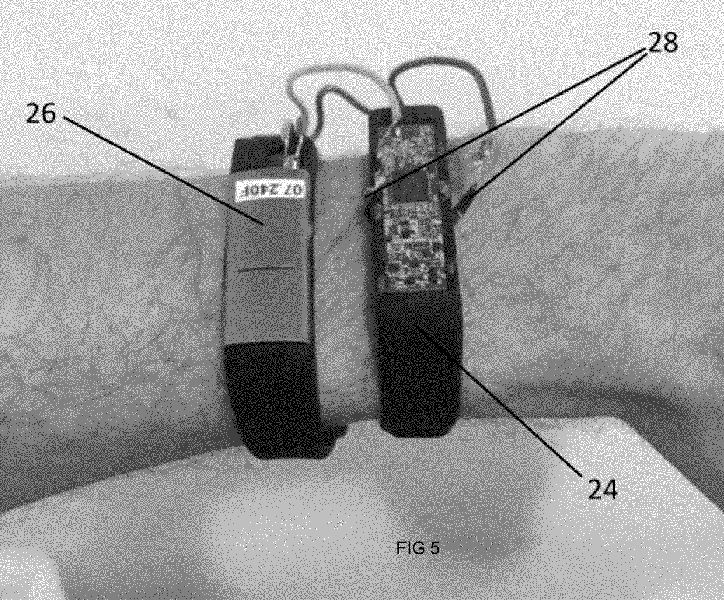

In FIG. 4, a photograph is shown of an embodiment of the invention seen from the skin-facing side. FIG. 5 is the same embodiment of the invention seen from the wrist side. The two skin proximity sensing electrodes 2 are made of copper tape in this embodiment, cut to shape using scissors to approximate a semi-circular geometry, and are shown attached onto a polypropylene plastic cartridge 16 using acrylic adhesive (shown schematically in FIG. 3 as 18). The electrodes are covered using an acrylic bioadhesive 20 (3M 1509). The cartridge 16 is attached to a dummy wristband 24, and the electrodes 2 are electrically attached to a second wristband 26 by means of two copper wires 28. In the second wristband 26 is a microcontroller that is used to record capacitance values by means of a PSOC 4 (Cypress), which further relays data via a Bluetooth Low Energy antennae to an iOS (Apple) smartphone. Data from the smartphone is relayed to a cloud-based server. This dual-wristband design is used to demonstrate the principle of a functional prototype, but a preferred embodiment combines the sensing electrodes 2, microcontroller 6 into just one wristband to reduce the physical size of the embodiment.

This preferred embodiment is shown schematically in FIG. 3. The skin proximity sensors 2 could alternately be used to perform an AC electrical impedance measurement, galvanoresistance, or other measurements familiar to those skilled in the art.

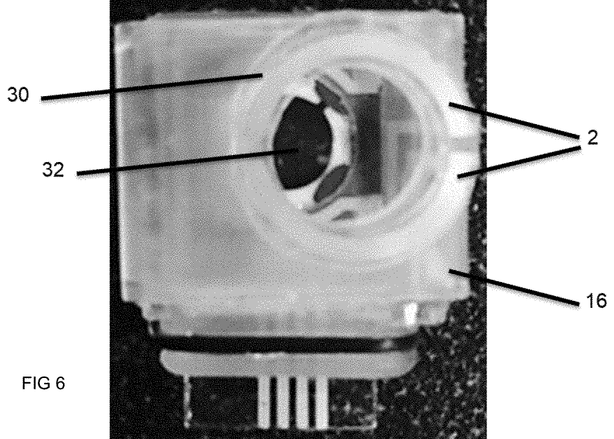

In one embodiment of the invention, two semi-circular and planar skin proximity electrodes 2 were screen-printed onto a PET substrate 30 (FIG. 6). The electrodes were adhered around a central port to the transdermal alcohol sensing electrode 32, all fitted into a cartridge 16 made of polypropylene, using a pressure-sensitive adhesive 18 familiar to those skilled in the art. The cartridge can attach to housing 4 that houses a microcontroller 6. The cartridge 16 can removably couple to the housing 4 by a sliding, clicking or similar mechanism familiar to those skilled in the art. In this embodiment, a disposable cartridge, for example containing alcohol oxidase enzyme for a transdermal alcohol sensing application, can be disposed of after use and replaced with a new cartridge.

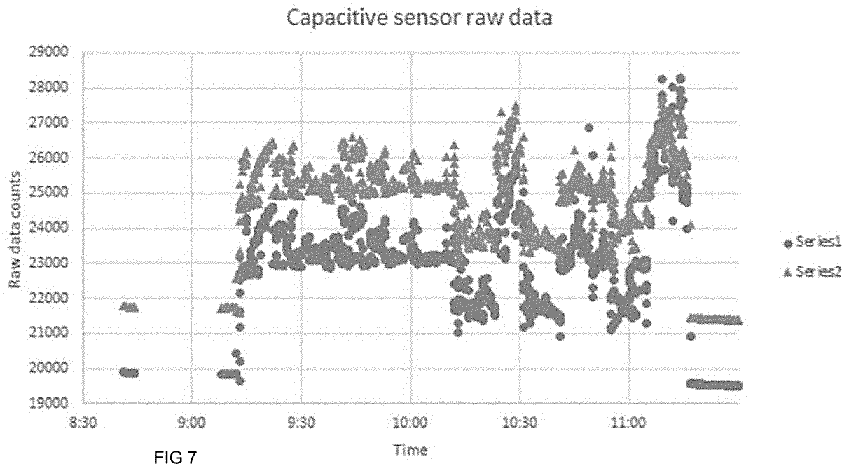

FIG. 7 shows a graph of the measured raw capacitance values of the reduced-to-practice embodiment described in FIGS. 4 and 5. The time begins at 9:00 and ends at 11:30. At 9:08, the wristband was connected and a baseline value was collected. At 9:11, the proximity sensing wristband was placed on a wrist. The sensors were worn continuously until 10:11, at which time a tamper event was simulated by attempting to wedge a thin piece of plastic between the skin and the sensor. The raw sensors signals do appear to decrease in magnitude. However, the raw capacitance proximity data is too noisy to be able to distinguish normal wear from a tamper event. The wristband is tampered with again at 10:24, 10:42, and removed completely at 11:17.

Therefore, the signal processing described below has been developed to significantly improve the signal-to-noise ratio.

Before the wristband is placed on the skin, two baseline values B1 and B2 are measured.

Using the data in FIG. 7, these values are B1=19831 and B2=21723 respectively. At the server, the two signals S1(t) and S2(t) are compared, and a ratio R is derived as follows:

$$R = \frac{( {S}_{1} - {B}_{1} )}{( {S}_{2} - {B}_{2} )}$$

The ratio R is nominally 1, but any asymmetry in capacitance between the two electrodes will cause the ratio to deviate significantly from unity. Alternately, values could be processed on the PSOC 4 microcontroller or the iOS app. Alternatively, a difference between signals could be used instead of a ratio, or some other formula to mathematically compare the two signals, familiar to those skilled in the art.

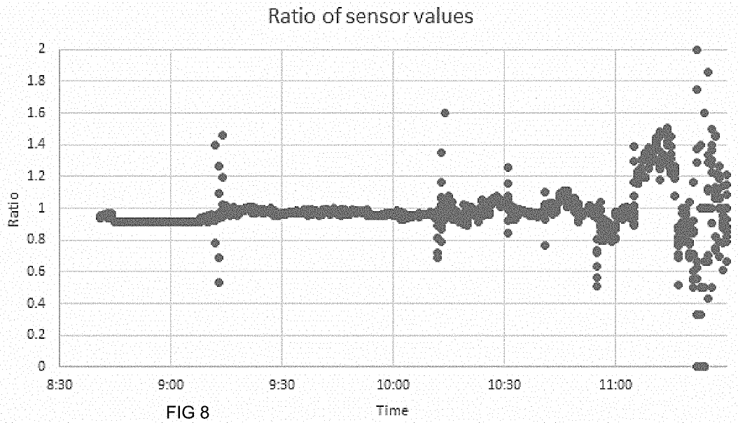

In FIG. 8, the ratio R is plotted as a function of time. As can be seen, R remains between 0.90 and 1.10 during normal wear. When the wearer attempted to insert a piece of plastic between the wrist and the capacitive sensor at 10:12, the ratio R deviated significantly from unity, reaching values of 0.68 and 1.6 during the tamper. Thus, a tamper event was flagged at 10:12.

The change in ratio R can be used to detect a tamper event, where a wearer attempts to insert an ethanol impermeable plastic between the skin and the sensor. The unexpected benefit to utilizing two sensors is that the ratio R is nearly insensitive to typical skin movement. Only upon tampering does the ratio deviate appreciably from unity.

At t=11:17, the wristband is removed from the wrist entirely. The ratio R temporarily deviates from unity, and the signal magnitude drops to approximately the baseline values B1 and B2.

Although the raw values can also indicate that the wristband has been removed entirely, the ratio R can detect both types of tampering: inserting objects, and removing the wristband.

In this embodiment, a bioadhesive was placed over the electrodes. One side of the bioadhesive is formed of a silicone bioadhesive that can be repeatedly removed from the skin. The bioadhesive serves two functions. First, the bioadhesive forms a seal with the skin which enables ethanol vapors that may be present to be detected by the sensor by preventing the escape of ethanol vapors into the environment. Secondly, the bioadhesive creates a well-definite interface between the skin and the sensor. The capacitive sensors used for proximity sensing read values that depend highly on the distance between the sensor and the skin. In the absence of a bioadhesive, normal wear of a wristband would likely create excessive noise. However, with the addition of a bioadhesive, the capacitive sensors are able to read values within the dynamic range of the capacitive readout circuitry without saturating it.

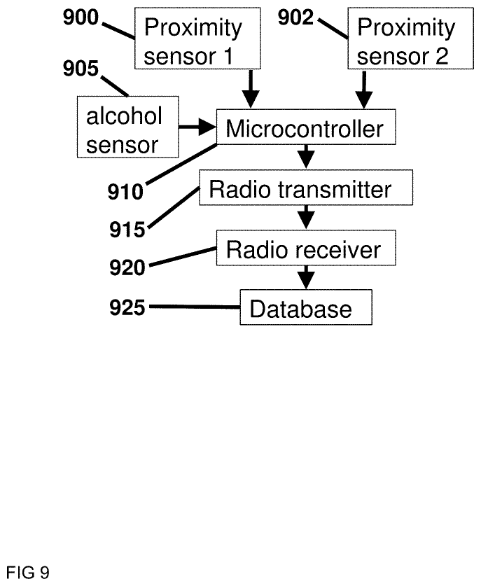

In FIG. 9, a flowchart illustrates an embodiment of the invention. Two proximity sensors 900, 902, for example electrically conductive electrodes, are attached to a microcontroller 910. An alcohol sensor 905, for example a transdermal enzymatic alcohol sensor, is also attached to the microcontroller 910, for example via analog front-end electronics. The microcontroller 910 converts the signals into digital signals via an Analog-to-digital converter. The signals are then sent via a radio transmitter 915, for example a Bluetooth Low Energy antenna at 2.45 GHz. A radio receiver 920 receives the signals, for example another Bluetooth Low Energy transceiver on an Apple iPhone smartphone. This smartphone then relays the data to a database 925, for example using a wireless wifi network, 4G network, or other means familiar to those skilled in the art.

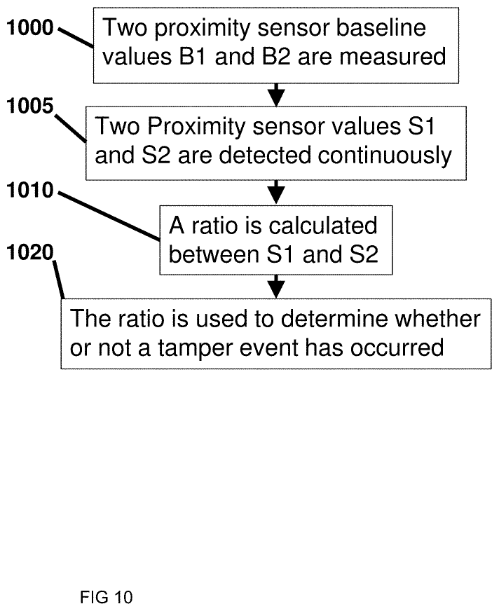

In FIG. 10, a flowchart of the method is described. First, two proximity sensor baseline values B1 and B2 are measured at 1000. Then, the sensors are placed on the skin and proximity sensor values S1 and S2 are detected continuously at 1005. A ratio is calculated using the sensor values S1 and S2 at 1010, together with the baseline values B1 and B2. Finally, the ratio is used to determine whether or not a tamper event has occurred at 1020.

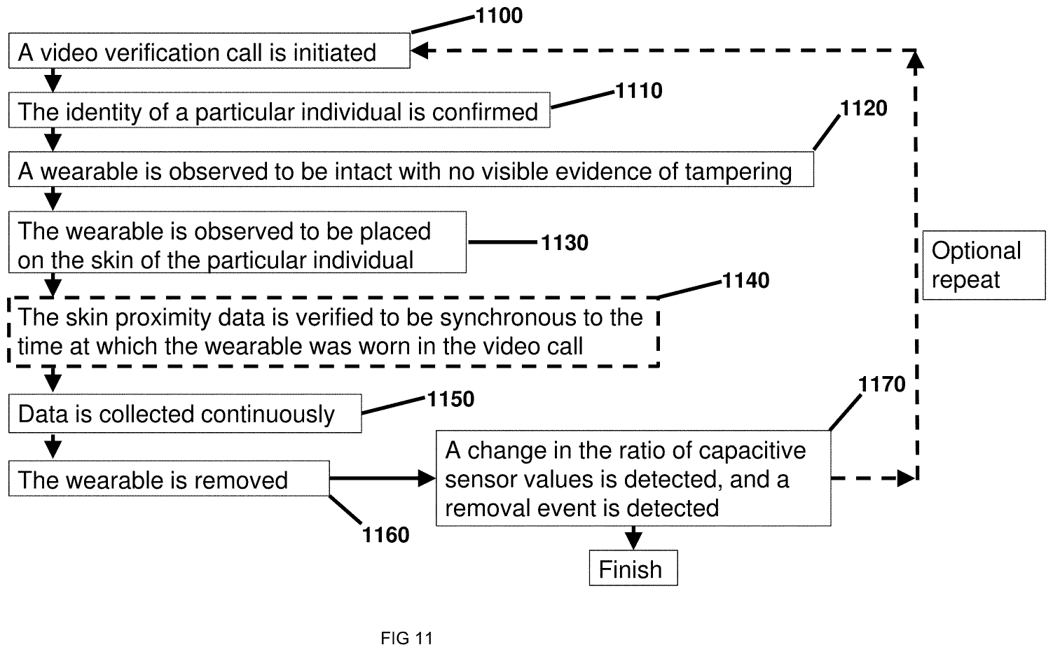

In another embodiment (see FIG. 11), within the application of tamper-proof alcohol sensing, a video check-in is used. The video check in may for example, be attended by either two people, or a person submitting a video, or a person chatting with an artificial intelligence software program. For example, the Zoom web video conferencing program can be used to initiate a call between a participant in an alcohol monitoring program with another person that is monitoring them at 1100. The identity of the person is validated at 1110. In the check-in, the presence of a cartridge is validated and examined for visible signs of tampering at 1120, for example ethanol impermeable plastic over the cartridge, or damage to the alcohol-sensing cartridge. Next, the wearable is observed to be placed on the correct individual at 1130 by visual inspection, for example examining the skin color, arm hair pattern, tattoos, other identifying features. If the cartridge is placed on the correct individual, a timestamp is noted, which defines the start of no-tampering monitoring. The time at which the wristband was placed on the individual can later be compared to the skin proximity sensor for additional validation at 1140. With this application, the ease of tampering is reduced. In this embodiment, the wristband may be removed for periods of time, for example for 1 hour while swimming, and then a subsequent inspection can be performed via video. This optional repeat allows for multiple interruptions to an otherwise continuous measurement process. This has particular advantages for people undergoing alcohol use disorder treatment, where swimming, going to the beach, doing dishes, working on an automobile, or other wet and dirty jobs could otherwise interfere with the wearable. Enabling continuous measurement with sporadic breaks is a big advantage over previous technology such as SCRAM, which must be worn continuously and thus are not compatible with many activities.

The flowchart also shows data being collected continuously at 1150. The wearable is removed 1160, and removable event is detected at 1170.

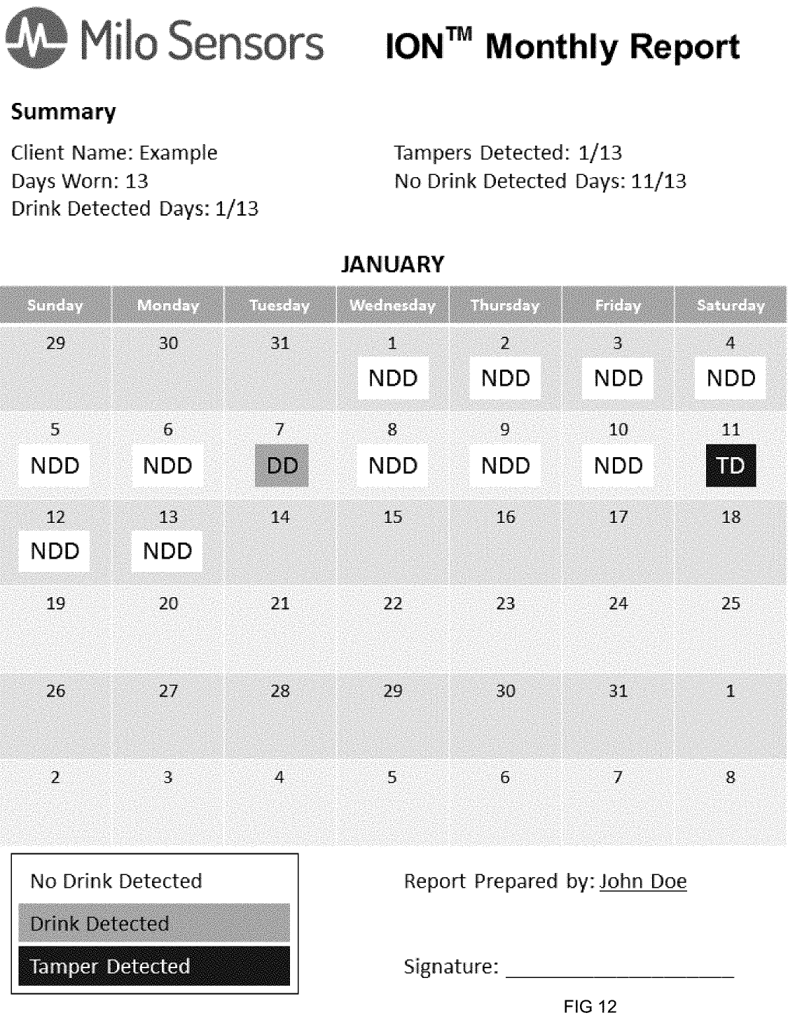

In one embodiment, the tamper data generated using skin proximity sensors is utilized to generate a calendar view (FIG. 12). In this way, a client, for example in an alcohol monitoring program, may be able to see a list of compliant days. The calendar can readily show on which days a tamper event was detected or alcohol was consumed, which can facilitate communication surrounding consumption of alcohol between clients, spouses, clinicians, coaches, sponsors, and any person that has access to the calendar.

In one embodiment, the skin-worn sensing device is in the form-factor of a wrist-worn wearable, in another it is an ankle-worn wearable, and in yet another it is in the form of a patch that can be placed anywhere on the body.

Although only a few embodiments have been disclosed in detail above, other embodiments are possible and the inventors intend these to be encompassed within this specification.

The previous description of the disclosed exemplary embodiments is provided to enable any person skilled in the art to make or use the present invention. Various modifications to these exemplary embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A sensing device comprising:
a housing, worn in a way to press a skin-interfacing surface of the housing against a skin of a user when the housing is being worn;
a first skin-proximity sensor and a second skin-proximity sensor, located on said skin-interfacing surface to be pressed against the skin of the user when the housing is being worn; and
a processing unit, operating to receive a first signal and a second signal respectively from the first skin-proximity sensor and the second skin proximity sensor, to analyze the first signal and the second signal, including comparing the first signal to the second signal, to detect tampering by the user.

2. The device of claim 1, where the skin proximity sensors are capacitive sensors.

3. The device of claim 1, further comprising a bioadhesive between the proximity sensors and the skin.

4. The device of claim 1, where the processing unit compares the first signal to the second signal to detect a ratio between the first signal and the second signal.

5. The device of claim 4, where the processing unit operates by obtaining baseline values of the first signal and the second signal before the housing is placed on the skin, obtaining sensor values of first signal and the second signal while the housing is on the skin, and comparing a first ratio between the baseline values with a second ratio between the sensor values, to detect tampering.

6. The device of claim 1, further comprising hardware forming a wireless connection, and where the processing unit wirelessly sends data via the wireless connection.

7. The device of claim 6, where the processing unit sends data to a cloud-connected server using a wireless or cellular network via the smartphone.

8. The device of claim 1, where the first skin-proximity sensors and a second skin-proximity sensor surround another sensor.

9. The device as in claim 8, wherein the another sensor is a transdermal alcohol sensor which detects alcohol vapors that are emitted from the skin.

10. The device of claim 9, where the first skin-proximity sensors and a second skin-proximity sensor are used to detect tampering with the alcohol sensor.

11. A method of tamper detection comprising:
obtaining a first skin proximity value, and a second first skin proximity value from sensors on a housing;
determining if a ratio between the first skin proximity value, and the second first skin proximity value deviates from unity by a specified amount; and
generating an indication of a tamper event if the ratio deviates from unity by the specified amount, and determining there is no tamper event if the ratio does not deviate from unity by the specified amount.

12. The method of claim 11, where the tamper events and no-tamper events are arranged into a calendar view.

13. A system for discontinuous sensing, comprising:
collecting a time-series of data removing wristband videos;
obtaining a video of a cartridge and a wrist of a user;
determining, from the video of the cartridge, if the cartridge has been tampered with;
observing the video to verify that the wrist belongs to a specified individual being monitored;
observing a time-series of sensor data to determine that the cartridge remained in the wristband during the time series;
generating a tamper signal or no tamper signal based on the data including the videos.
